(19) United States
(12) Patent Application Publication
(10) Pub. No.: US 2012/0258418 A1
(43) Pub. Date: Oct. 11, 2012

(54) Portable Simultaneous Tooth Prophylaxis And Bleaching System

(76) Inventor: Feimo Shen, Milpitas, CA (US)

(21) Appl. No.: 13/079,817

(22) Filed: Apr. 5, 2011

(51) Int. Cl. A61C 19/06; A61B 1/24

(52) U.S. Cl. 433/29; 433/122; 433/82

(57) ABSTRACT

A portable dental prophylaxis and bleaching system is provided for simultaneous tooth polishing and bleaching. It includes an oscillatory driving handpiece, a dental angle with a disposable rubber cup assembly, a dual-component dispenser, and a disposable mouthpiece. The battery-powered handpiece transforms a continuous rotation through a driving cam into a rotary oscillation which is then transferred through the attached dental angle to the rubber cup with an obtuse angle at its distal output end. The dispenser is operated manually to correctly apportion and mix the dual treatment materials into the rubber cup. In order to provide longer shelf life and effective bleaching action, the treatment materials are maintained at optimal pH values in separate compartments within the dispenser before use. The disposable mouthpiece is shaped for easy access to the user's teeth while protecting the gum surfaces. The system can be used both in dental clinics and at home.

### CROSS-REFERENCE TO RELATED APPLICATIONS

U.S. Patent Documents

| Patent | | | Current U.S. |
|---|---|---|---|
| Number | Published Date | Inventors | Class |
| 7,762,813 | Jul. 27, 2010 | Seals et al | 433/124; 433/125 |
| 7,601,002 | Oct. 13, 2009 | Milanovich | 433/215 |
| | | et al | |
| 7,255,559 | Aug. 14, 2007 | Shen et al | 433/125; 433/118; |
| | | | 433/166 |
| 7,160,111 | Jan. 9, 2007 | Baughman | 433/216; 433/140; |
| | | | 433/29; 600/242 |
| 7,156,659 | Jan. 2, 2007 | Pernot | 433/144; 433/133 |
| 7,153,133 | Dec. 26, 2006 | Chia et al | 433/125 |
| 6,916,176 | Jul. 12, 2005 | Schatz et al | 433/126; 403/320 |
| 6,294,155 | Sep. 25, 2001 | Thomas et al | 424/49; 423/339 |
| 6,247,931 | Jun. 19, 2001 | Postal et al | 433/118; 433/122; |
| | | | 433/125 |
| 6,174,516 | Jan. 16, 2001 | Curtis et al | 424/53; 424/613; |
| | | | 424/616 |
| 6,168,433 | Jan. 2, 2001 | Hamlin | 433/125 |
| 5,928,628 | Jul. 27, 1999 | Pellico | 424/49; 424/53 |
| 5,902,107 | May 11, 1999 | Lowell | 433/130; 433/112; |
| | | | 433/125 |
| 5,749,728 | May 12, 1998 | Bailey | 433/125; 433/126 |
| 5,571,012 | Nov. 5, 1996 | Witherby | 433/125; 433/126; |
| | | et al | 433/133 |
| 5,531,599 | Jul. 1, 1996 | Bailey | 433/125; 433/126; |
| | | | 464/181 |
| 5,503,555 | Apr. 2, 1996 | Bailey | 433/126; 433/125 |
| 5,433,605 | Jul. 18, 1995 | Strobl | 433/112; 433/125; |
| | | | 433/126; 433/130 |
| 5,423,679 | Jun. 13, 1995 | Bailey | 433/125; 433/126 |
| 5,374,189 | Dec. 20, 1994 | Mendoza | 433/125; 433/132 |
| 5,328,369 | Jul. 12, 1994 | Bailey | 433/125; 433/126 |
| 5,139,421 | Aug. 18, 1992 | Verderber | 433/31; 433/30 |
| 5,074,788 | Dec. 24, 1991 | Nakanishi | 433/115; 433/129 |
| 4,681,540 | Jul. 21, 1987 | Landgraf | 433/126; 422/131; |
| | | et al | 422/29; 433/131 |
| 4,648,838 | Mar. 10, 1987 | Schlachter | 433/29; 433/126; |
| | | | 433/80 |
| 4,460,337 | Jul. 17, 1984 | Landgraf | 433/29 |
| | | et al | |
| 4,382,790 | May 10, 1983 | Loge et al | 433/126 |
| 2010/0035205 | Feb. 11, 2010 | Wang et al | 433/133 |
| 2006/0127844 | Jun. 15, 2006 | Michaelian | 433/125; 433/84 |
| 2005/0050658 | Mar. 10, 2005 | Chan et al | 15/22.1; 433/29 |
| 2003/0180688 | Sep. 25, 2003 | Vocaturo | 433/215; 433/80 |
| | | et al | |
| 2001/0046477 | Nov. 29, 2001 | Wolfe | 424/53 |

### FIELD OF THE INVENTION

The present invention relates to dental cleaning and bleaching. The invention is a battery-powered dental tool that effectively cleans and at the same time bleaches teeth. It is an assembly that has a driving handpiece, a dental angle, and a disposable rubber cup. For longer shelf life and effective action during use, the present invention also has an apparatus that mixes two properly pH-valued materials at the moment they are dispensed. By applying the mixed prophy and bleaching materials on teeth, the movement of the rubber cup polishes and bleaches simultaneously. To optimally and quickly achieve clean and white teeth, the invention employs a mixture of materials with bleaching capability that is pH and temperature optimized for oral use. The present invention uses a handpiece to transfer the continuous rotation of an electric DC motor into a rotary oscillation through a specialized cam bar, so as to drive the dental angle with this same rotary oscillation. Moreover, a disposable mouthpiece is used during operation in order to protect gum surfaces from contact with the bleaching materials.

### BACKGROUND OF THE INVENTION

Conventionally, dental polishing with prophy paste and dental whitening with bleaching paste are done as two separate procedures in dental treatment. This sequential polishing and bleaching is time-consuming; therefore, a method of combining the two procedures is desirable. The background analysis can be separated into two parts: mechanical tool design and prophy/bleaching compound design.

A. Mechanical Tool Design

A conventional prophy angle is used to remove plaque and to polish the dentin surface of a tooth. A prophy cup is secured to the prophy angle and is rotated by a driving torque from a motor in a dental tool, such as a dental handpiece. A typical drive mechanism is a gear connection between a driving rotor gear and a driven rotor gear at a right angle. The rotation is thereby transferred from the driving dental tool to the prophy cup.

This prophy method has several drawbacks. First, the gear connection between driving and driven rotors is costly to manufacture. As disposable inexpensive plastic parts, gears are quickly worn out which results in bad engagement and operating noise. Second, the gear connection between driving and driven rotors usually transfers continuous rotation which splatters the prophy paste during operation, as opposed to oscillatory rotation which reduces splattering. Third, the typical gearing between driving and driven rotors is not suitable for an obtuse angle connection. An obtuse angle makes users feel more comfortable because it allows the user to maintain a correct neutral wrist position.

Previous patents have been published which describe improvements in the gear mechanism or methods to achieve operation at angles other than 90 degrees, such as U.S. Pat. 2006/0127844, U.S. Pat. Nos. 7,762,813, 7,255,559, 6,916,176, 6,247,931, 6,168,433, 5,902,107, 5,749,728, 5,571,012, 5,531,599, 5,503,555, 5,433,605, 5,423,679, 5,374,189, 5,328,369, 5,074,788, 4,681,540, 4,460,337 and 4,382,790. However, in the present market, the products from those patents exhibit cost weakness or noise problems, typically because of the complicated structure of the non-gear mechanism.

U.S. Pat. No. 7,153,133 discloses a transmission assembly which uses multiple linkage shafts to connect a driving shaft and a head mount, both with multiple mounting holes. Each linkage shaft rotates and slides in the corresponding mounting holes in the driving shaft and the head mount. It solves several drawbacks of the gear-transfer prophy angles, but the small mounting holes on the driving shaft and head mount make the assembly time-consuming. Furthermore, because of the size limitation of the driving shaft and head mount, the torque arm of the linkage shafts is small, and the driving torque is reduced even more by the friction from the fast movement of the driving shaft and head mount pressing on the inner surfaces of the housing.

B. Prophy/Bleaching Compound Design

One conventional tooth whitening method is to place peroxide upon a patient's teeth. Carbamide is a compound that rapidly releases hydrogen peroxide. The oxidation-reduction reaction of peroxide bleaches the enamel of the teeth. A popular application of this method is to place a carbamide-bearing compound in a dental tray and apply the tray to the patient's teeth for a length of time ranging from minutes to overnight.

This whitening method has several drawbacks. First, peroxide is an irritant. When peroxide comes into a long contact with the gums, it causes irritation or pain. Second, the mere placement of peroxide on the patient's teeth is not effective if the enamel rods of the teeth are closed. Only when they are open, can the enamel rods take up peroxide significantly.

One method of facilitating the opening the enamel rods during bleaching is acid etching. When an acid of sufficient concentration is applied to the teeth, the chemical action of the acid serves to open the enamel rods of the teeth. However, this method is potentially harmful to the gums and is also time consuming. Another method involves the use of a pre-mixed carbamide and dental abrasive agent mixture. Such a mixture is applied to the teeth and is then burnished onto the teeth. The dental abrasive agent in the prophy paste serves to abrade the teeth, accomplishing three objectives: 1) it opens the enamel rods to facilitate their uptake of the peroxide; 2) it removes stains from the tooth enamel through a mechanical scrubbing action; and 3) it polishes the tooth enamel through a mechanical buffing action. To decrease tooth sensitivity during bleaching, fluoride can be included in the dental abrasive agent. However, a disadvantage of this method is that it delivers weakened carbamide to the teeth, resulting is less effective whitening. In addition, most readily and economically available carbamides are highly unstable, losing much of their oxygen (thus being reduced) when exposed to air and when mixed with other ingredients such as prophylaxis paste for an extended period of time. Therefore, the effectiveness of pre-mixed peroxide as a tooth whitener is limited.

To overcome these drawbacks, previous patents have been published. However, those patents either use pre-mixed compounds and other less effective approaches such as U.S. Pat. 2005/0050658, U.S. Pat. Nos. 7,601,002, 6,294,155, 6,174,516, 5,928,628, or do not provide a cost-effective instant mixing method during usage, such as 2001/0046477.

U.S. Pat. Nos. 4,648,838 and 5,139,421 etc. use a light to warm up the bleaching materials to achieve an effective bleaching activation. However, the lights in those patents are stationary and are applied simultaneously to multiple teeth so that the user must remain still during the treatment. The structure of the lights is also complicated and costly.

US patent 2003/0180688 and U.S. Pat. No. 7,160,111 etc. use a mouthpiece to protect the gum surfaces from the bleaching materials on the teeth. However, with the mouthpiece of those patents, the user cannot simply leave the mouth open in a proper attitude without manual adjustment.

### BRIEF SUMMARY OF THE INVENTION

There are seven major aspects of this invention that address the drawbacks of current technology.

The first aspect is the rotary oscillation of the dental angle. Rotary oscillations reduce paste splatter during operation. In the invention, a continuous rotation of the motor output shaft is converted into a back-and-forth swinging action and then to a rotary oscillation through a rotary oscillation driving cam with a specially designed cam curvilinear slot. The cam curvilinear slot achieves an even swinging movement with a nearly equal time for both the rightward and leftward parts of this movement. Moreover, the cam curve results in a momentary pause in the back-and-forth swinging action at the point where it changes swinging direction so that the impulse from the motor shaft to the driving cam is reduced, thereby increasing the working life of the motor shaft and the driving cam.

The back-and-forth swinging movement is transferred as a rotary oscillation to a dental angle from the rotary oscillation driving cam via a rack-and-pinion gear connected to a drive shaft and thence through a slot-rod matching construction at the handpiece output end. The matched slot-to-rod connection is achieved, at the time of assembly, by inserting the bent driving rods into the corresponding slots on the conic nose end of the driving shaft. To avoid any possible mismatch between slots and rods, the diameter of the slots is gradually and smoothly enlarged at the conic nose end.

The second aspect is the transfer of the rotary oscillation through an obtuse angle using two rotors connected by multiple bent driving rods within the dental angle. This rotary oscillation is further transferred from the handpiece output end to a disposable rubber cup assembly which is firmly attached to the output end of the dental angle. The bent driving rods can be easily pressed into the mounting slots on the in-line rotor and the angled rotor thereby avoiding the time-consuming process of inserting the rods through end holes of these slots. During operation the bent driving rods dynamically touch the inner cylindrical surface of the dental angle housing as a bearing mechanism to reduce the friction between the rotors and the inner cylindrical surfaces of the housing. Moreover, the axes of the input-end and output-end are engaged at an obtuse angle so that the teeth prophylaxis and bleaching operation can be done more comfortably. This is because an obtuse angle bend in the body of the angle allows the user to maintain a neutral wrist position.

A third aspect is that the disposable rubber cup and rotor-cup adapter are pre-assembled or molded as a single entity which can be easily snapped onto and removed from the output end of the dental angle body. This means that the dental angle main body can be used repeatedly to reduce cost as a personal device at home.

A fourth aspect is that the dental angle housing consists of a housing body and a housing head cap. The housing head cap has a circular ring at the lower end. The housing body has a bent output end with an obtuse angle so that the upper end of the head cap matches the bent end of the housing body and snaps on to form an obtuse dental angle. The integral circular wall at the input end of the housing body and the circular ring of the housing head cap tightly holds the bent driving rods to the rotors during operation to reduce noise and wear.

A fifth aspect is a dual-component dispenser that proportionally mixes the prophy and bleaching compound immediately before tooth cleaning The dispenser comprises a central tube and an outer shell that are co-axial with the dispenser housing, a double piston with a central circular cross-section and a surrounding annular cross-section, a matched platform at the tail, and a disposable cap at the dispending end. High pH prophy paste and low pH bleaching paste are stored in the tube and shell, respectively during storage and transport. The dimensions of the central tube and shell have the proper volumetric proportion so that the compounds from the two compartments can be easily mixed to reach the correct pH value for use. In addition, the end cap serves both to seal the dispenser and as a mixing container after the pastes are dispensed. This invention allows long-term storage of bleaching compound at low pH, increasing the pH just at the moment of cleaning and bleaching. The higher pH opens the enamel rods at the surface of the teeth, resulting in a better bleaching effect.

A sixth aspect of the invention is a disposable mouthpiece for protecting gum surfaces during bleaching. The mouthpiece has a closed, curved edge that pushes the lips apart to expose the frontal teeth. The edges of the mouthpiece abut the edges of the roots of the teeth. The mouthpiece has a wedge-shaped positioning bite tab at both sides so that when the user bites down, the mouthpiece remains stationary within the mouth and the upper and lower lips are held open comfortably in a proper orientation.

A seventh aspect of the invention is a light bulb, such as an emitting diode (LED), mounted at a proper location on the handpiece housing so that the emitted light is focused directly onto the area near the rubber cup of the dental angle during operation. The light radiation heats up the bleaching material to speed up the bleaching action while providing illumination.

Other details and features of the invention will pointed out hereinafter.

### BRIEF DESCRIPTION OF THE DRAWINGS

The invention will be further described in conjunction with the attached drawings:

FIG. 1 shows the major subassemblies and the mouthpiece of the tooth prophylaxis and bleaching system. The subassemblies are the battery-powered handpiece, the dental angle, the rubber cup, and the paste dispenser. The mouthpiece is a one-piece component.

FIG. 2 is a cross-sectional view of the battery-powered driving handpiece with the dental angle and rubber cup assembled.

FIG. 3 shows the battery-powered driving handpiece of FIG. 2.

FIG. 4 is an axial cross-sectional view (a) and a longitudinal cross-section view (b) of the mechanism which transforms continuous rotation to oscillating rotation inside the handpiece housing.

FIG. 5 is a set of axial cross-sectional views showing the four quadrants of the cam movement as driven by the motor shaft, which illustrates how the cam transfers continuous circular rotation (clockwise, as viewed) of the motor shaft to a back-and-forth swinging movement—(a) extruded off-axis post starting into the first quadrant, (b) off-axis post starting into the second quadrant, (c) off-axis post starting into the third quadrant and (d) off-axis post starting into the fourth quadrant.

FIG. 6 is a cross-sectional view showing the output side of the rotary oscillation driving cam; how the rack-and-pinion gearing connection transfers the back-and-forth swinging movement of the cam to an oscillating rotation of the driving shaft.

FIG. 7 is a perspective view of the driving shaft which, when assembled, is located at the output end of the handpiece.

FIG. 8 shows the nose end of the driving shaft of FIG. 7 with two representative cross-sectional views at the nose and the body.

FIG. 9 is a cross-sectional view of the dental angle housing with the bent driving rods, the in-line rotor, the angled rotor, and the rotor-cup-adapter all assembled. It also shows the rubber cup assembled as well as the handpiece driving shaft inserted.

FIG. 10 is similar to FIG. 9 but without the rubber cup assembly and the handpiece driving shaft.

FIG. 11 is a perspective view of the bent rod driving mechanism.

FIG. 12 is a front cross-sectional view and corresponding longitudinal cross-sectional view of both the in-line rotor and the angled rotor showing the placement of the bent driving rods in the rotors.

FIG. 13 shows cross-sectional views of the angled rotor and the rotor-cup adapter with the rubber cup attached.

FIG. 14 is a perspective view of the angled rotor and the matching rotor-cup adapter.

FIG. 15 is a perspective view of the dental angle housing with the hollow housing body and the housing head cap assembled.

FIG. 16 shows a perspective view of the hollow housing body of the dental angle housing, similar to that of FIG. 15, but without the housing head cap.

FIG. 17 is a perspective view of the housing head cap of the dental angle housing.

FIG. 18 is a perspective view (partially cross-sectional) of the two-component paste dispenser.

FIG. 19 shows an axial cross-sectional view, on the left, and a longitudinal cross-section view, on the right, of the central tube of the two-component paste dispenser of FIG. 18.

FIG. 20 shows axial and longitudinal cross-section views of the double piston of the two-component paste dispenser of FIG. 18.

FIG. 21 is a perspective view of the mouthpiece.

### DETAILED DESCRIPTION OF THE INVENTION

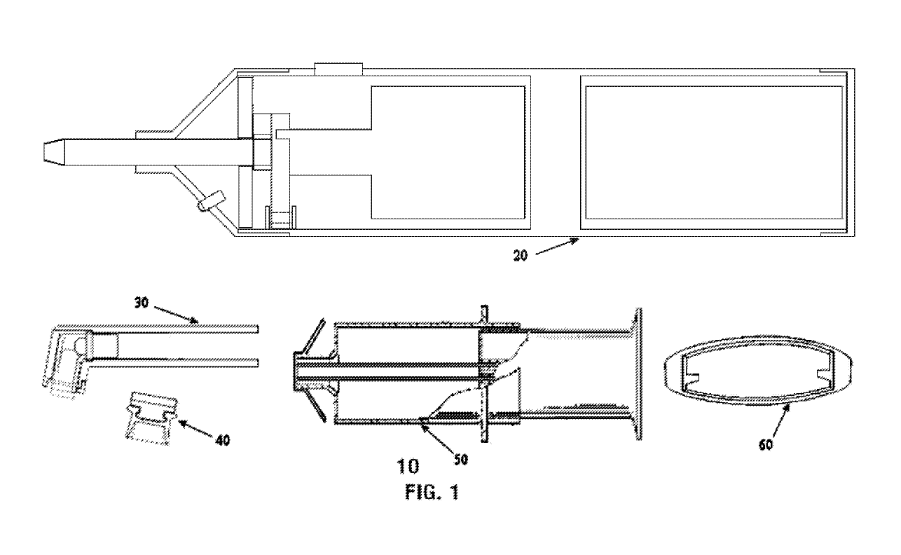

FIG. 1 shows the system components of the tooth prophylaxis and bleaching system 10 which consists of a battery-powered driving handpiece 20, a dental angle 30, a rubber cup and rotor-cup adapter 40, a dual-component paste dispenser 50, and a protecting mouthpiece 60.

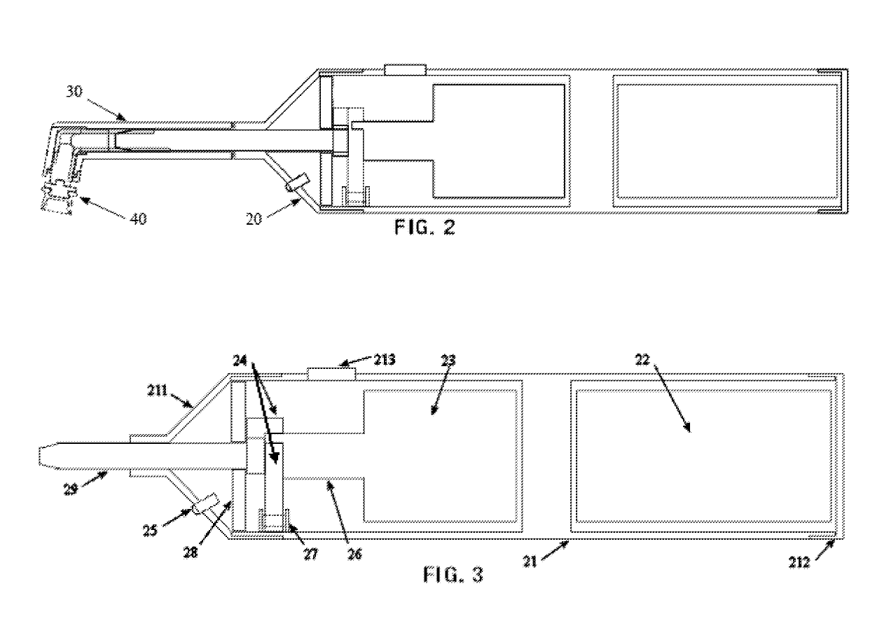

FIG. 2 shows a cross-sectional assembled view of the battery-powered driving handpiece 20 including the dental angle 30 with the rubber cup assembly 40.

FIG. 3 shows the parts inside of the handpiece 20: a battery chamber 22, a direct current (DC) electric motor 23 with an output rotating shaft 26, an on-off switch 213, a driving cam 24 with a rack and pinion connection to a driving shaft 29 which transfers the output of the cam to the dental angle, a driving shaft positioner 28 whose central hole holds the driving shaft 29 axially, an light bulb 25, and a basic housing 21 with a housing output cap 211 and a housing battery cap 212 to enclose all the parts.

FIG. 4 shows the transformation mechanism inside the handpiece 20 in an axial cross-sectional view (a) and a longitudinal cross-sectional view (b). The mechanism transforms a continuous rotation into an oscillating rotation via a cam setup. The motor shaft disk 261 of the electric motor 23 rotationally drives the cam 24 via the extruded off-axis post 262. The driving cam 24 is swung around the cam pivot post 271 which is fixed on the extruded cam pivot on housing 27 of the handpiece basic housing 21. The cam rack 241 is molded with the driving cam 24 as an entity, so that when the driving cam 24 swings, the cam rack 241 swings with it. Hence a back-and-forth swinging movement is transformed into an oscillating movement of the driving shaft 29 as the cam rack teeth 242 repeatedly pass over the driving shaft pinion 291.

There are two stages of motion transfer: 1) continuous rotation to swinging, 2) swinging to oscillating rotation. The continuous rotation of the motor shaft 26 and the oscillating rotation of the driving shaft 29 are coaxial along the central axis of the handpiece basic housing 21, but the back-and-forth swinging movement of the driving cam 24 rotates around the cam pivot post 271 which is located on the inner wall of the housing.

FIG. 5 shows the first stage: motor to cam. The power input comes from the electric motor 23. The output is the driving cam 24 that has a center of rotation about the cam pivot post 271 secured to the inner wall of the cylindrical handpiece basic housing 21. The motor shaft 26 has a motor shaft disk 261 firmly connected to the axle of the motor 23 (refer to FIG. 4) that rotates concentrically. The motor shaft disk 261 has an extruded off-axis post 262 that inserts into a cam curvilinear slot 240 in the driving cam 24. The continuous rotation of the off-axis post 262 about the axis of the motor shaft 26 forces the driving cam 24 to be swung back and forth. This swinging movement can be understood by considering the four quadrants of a motor shaft's revolution. First quadrant as shown on FIG. 5(a): the cam curvilinear slot 240 on the driving cam 24 is contoured such that when the off-axis post 262 moves clockwise a quarter of a circle (starting from the top), it pushes the right side 245 of the cam curvilinear slot 240 so that the driving cam 24 is swung to the right side around the cam pivot post 271. Second quadrant as shown on FIG. 5(b): the off-axis post 262 continues to move clockwise down along the contour of the cam curvilinear slot 240 which causes the cam 24 to experience a short pause in its motion, after which the off-axis post 262 contacts the left side 246 of the cam curvilinear slot 240 so that the driving cam 24 changes direction and begins to swing to the left side. Third quadrant as shown on FIG. 5(c): the off-axis post 262 moves from the bottom upward another quarter of circle, continuously pushing the driving cam 24 and swinging it to the left side around the cam pivot post 271. Fourth quadrant as shown on FIG. 5(d): the off-axis post 262 continues clockwise up another quarter of a circle and returns to the top of the curve, which causes the cam 24 to experience a short pause, after which the off-axis post 262 contacts the right side 245 of the cam curvilinear slot 240 so that the driving cam 24 changes direction again and starts to swing to the right side, finishing a cycle. This back-and-forth swinging movement of the driving cam 24 around the cam pivot post 271 is repeated once per revolution of the electric motor 23. The specialized cam curvilinear slot 240 of the driving cam 24 in this invention achieves an approximately even swinging movement with approximately equal amounts of time spent moving right and left. It improves upon the weakness of the conventional straight slot cam that causes the rightward swing to take much longer than the leftward swing (assuming clockwise rotation, as drawn).

If the motor shaft 26 rotates in the counter-clockwise direction, the roughly even swinging movement, with the pauses at the extremes of the cam motion, is achieved similarly except that all the rotating directions are opposite to those described above. With this design, balanced swinging movement with pauses at the extremes is achieved independent of the rotation direction of the electric motor 23.

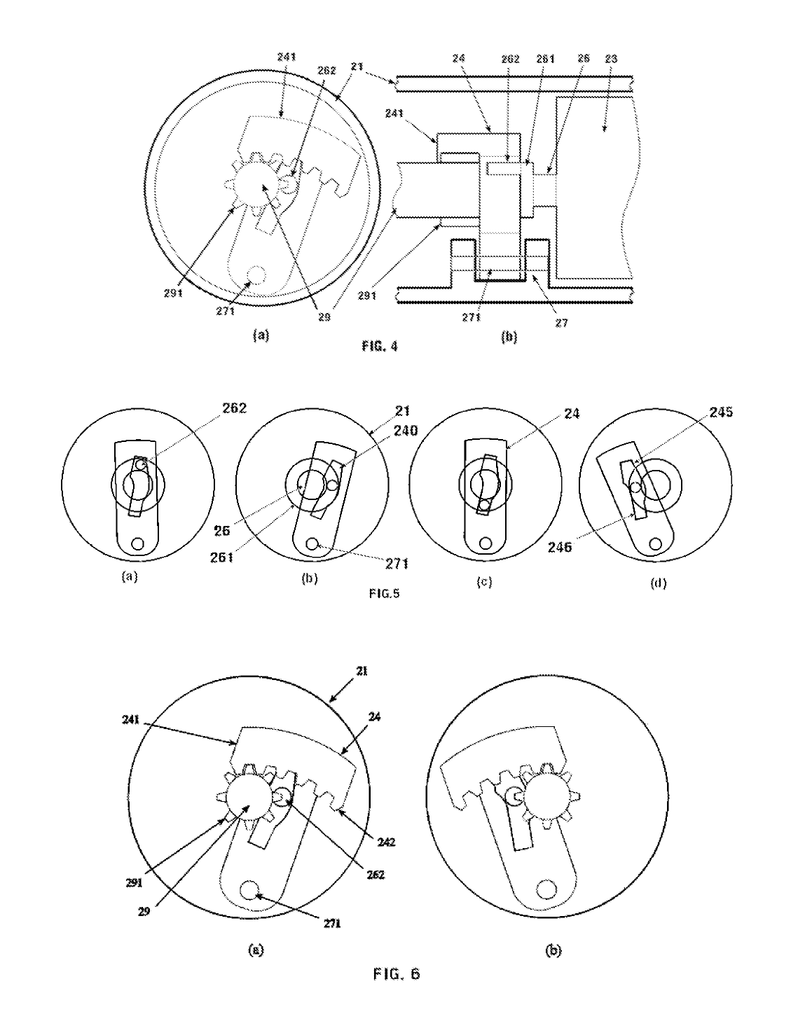

FIG. 6 shows the second stage of motion transfer: driving cam 24 to driving shaft 29. The input is the driving cam 24 and the output is the driving shaft 29. The back-and-forth swinging movement is transferred to the driving shaft 29 by a rack-and-pinion gearing connection. The torque source is the cam rack 241 which is located at the top of the driving cam 24 (with the teeth on the inner edge). The cam rack 241 is combined with the driving cam 24 to form a single entity. The driving shaft 29 has a circular driving shaft pinion 291 at its input end that matches the cam rack 241 on the driving cam 24. The central axis of the driving shaft 29 lies along the central axis of the handpiece 20. Hence the swinging movement of the driving cam 24 is transferred to a matched rotary oscillation of the driving shaft 29. FIG. 6(a) shows the location of the cam 24 when it is swung to the extreme right of its motion around the cam pivot post 271. From the location of the cam 24 in FIG. 6(a) to the location of the cam 24 in FIG. 6(b), the driving shaft 29 rotates clockwise around its axis by an angle of almost 180 degrees. The driving shaft's other end connects to the dental angle 30 via a matched slot and rod connection structure described below.

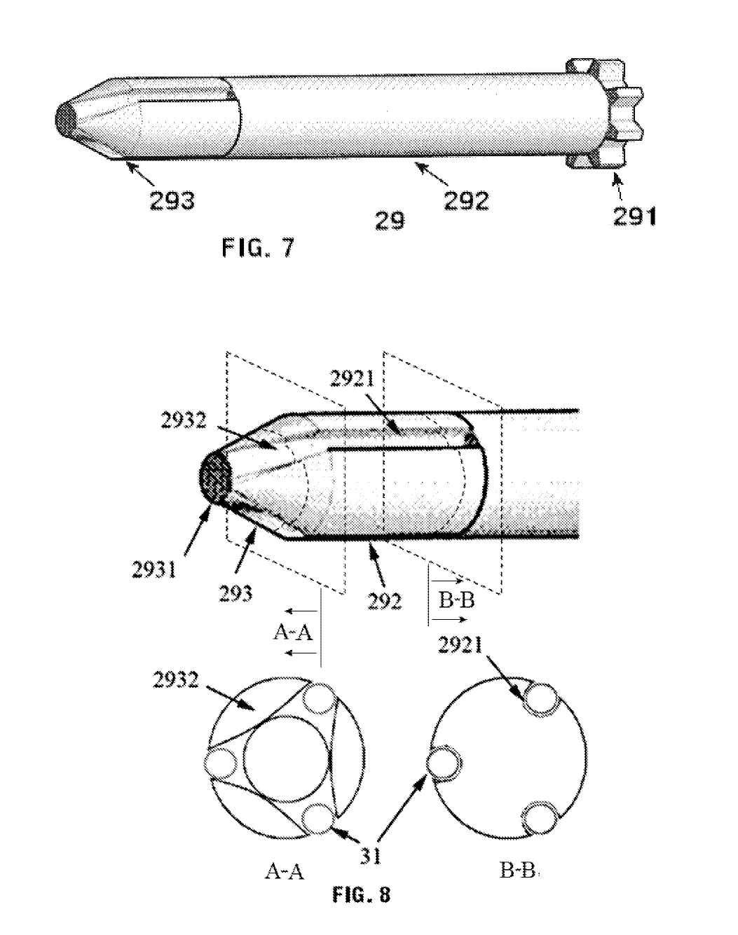

FIG. 7 shows a perspective view the driving shaft 29 of the driving handpiece 20. The circular driving shaft pinion 291 is attached to the driving shaft 29 at the input end of the driving shaft body 292. The driving shaft pinion 291 matches the cam rack teeth 242 on the driving cam 24 (refer to FIG. 6). After assembled, the driving shaft positioner 28 (refer to FIG. 3) positions the driving shaft body 292 axially by its central hole. A conic driving shaft nose 293 is located at the output end of the driving shaft body 292.

FIG. 8 shows a detailed perspective view of the conic driving shaft nose 293 of the driving shaft 29 shown in FIG. 7 with its cross-sectional views cut at locations A-A and B-B. The driving shaft nose 293 loosely fits into the input end of the housing 34 of the dental angle 30 when assembled. The number of driving shaft slots 2921 on the cylindrical surface of the driving shaft body 292 is matched to the number of the bent driving rods 31 of the dental angle 30. The diameter of the shaft nose end 2931 is smaller than the diameter of the driving shaft body 292. The driving shaft nose 2931 has a conic shape so that the front end 2931 is easily insertable into the dental angle 30. During the insertion of the dental angle 30 into the driving handerpiece 20, the shaft nose slope 2932 guides the driving rods 31 into the driving shaft slots 2921.

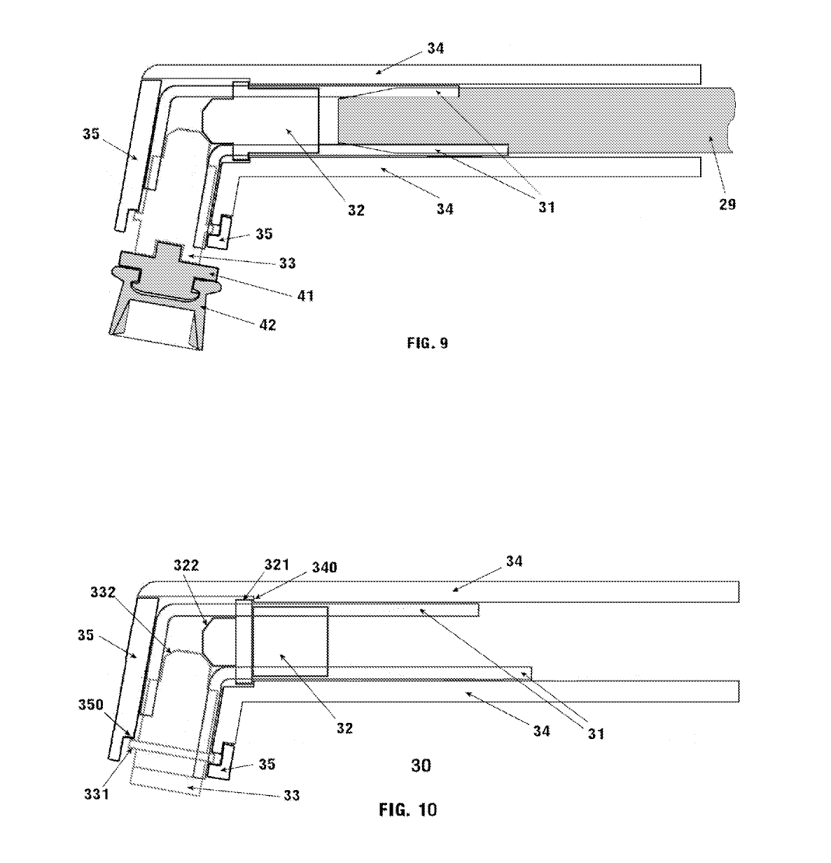

FIG. 9 shows a cross-sectional view of the dental angle housing with the bent driving rods 31, the in-line rotor 32, the angled rotor 33, and the rotor-cup adapter 41 inside, as well as with the rubber cup 42 assembled and the handpiece driving shaft 29 inserted. After assembly, the dental angle housing, which consists of the main hollow housing 34 and the angled housing head 35, keeps the bent driving rods 31, the in-line rotor 32, and the angled rotor 33 together as an integral piece. The rotary oscillation is transmitted from the in-line rotor 32, via the bent driving rods 31, to the angled rotor 33, and then to the assembled or molded cup assembly 40 of the rotor-cup adapter 41 and the rotor-cup 42.

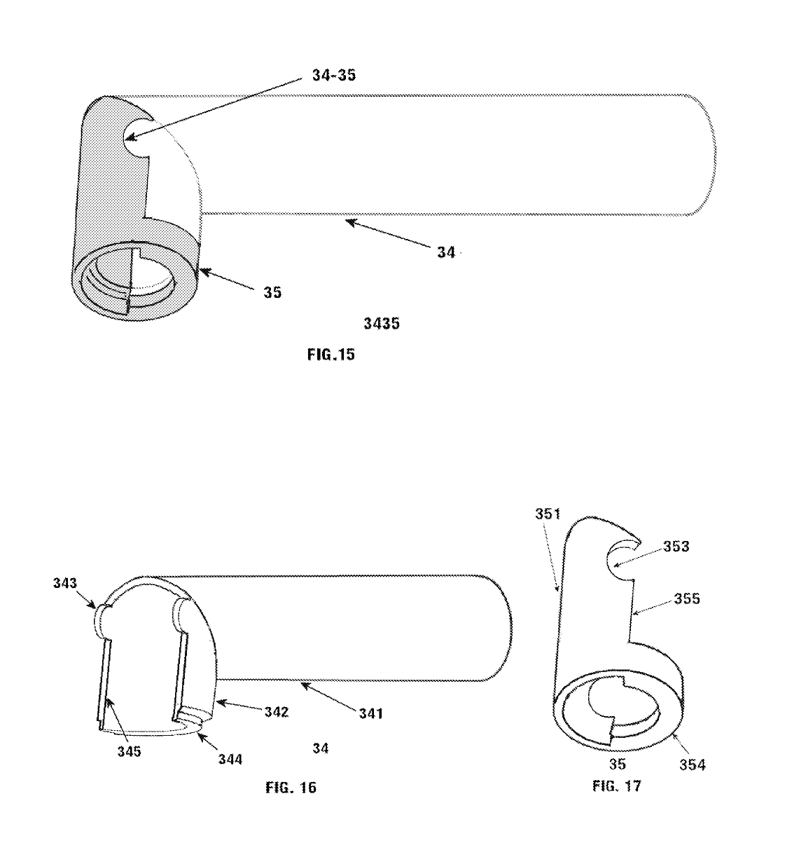

FIG. 10 is similar to FIG. 9 but without the assembled or molded cup assembly 40 of the rotor-cup adapter 41, the rubber-cup 42, and the handpiece driving shaft 29. There exists an in-line rotor flange 321 near the in-line rotor conic head 322 of the in-line rotor 32, and another angled rotor flange 331 near the output end of the angled rotor 33. The in-line rotor flange 321 maintains the axial position of the in-line rotor 32 through contact with the positioning step 340 in the inner tube of the main hollow housing 34. Similarly, referring to FIG. 16 and FIG. 17, the angled rotor flange 331 maintains the axial position of the angled rotor 33 through contact with the bottom step 344 on the angled outlet 342 of the main hollow housing body 34 and through contact with the angled housing step 350 on the angled housing head cap 35. The conic head 322 of the in-line rotor 32 and the conic head 332 of the angled rotor 33 contact each other when the components are assembled inside the dental angle housing 3435, which is also necessary to maintain the axial position of the rotors 32 and 33.

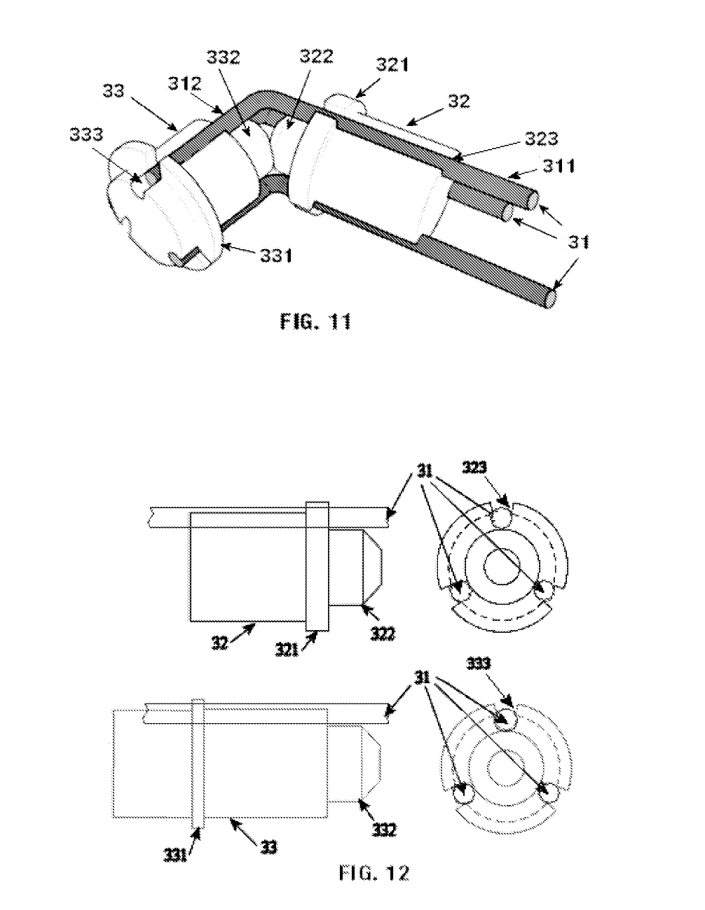

FIG. 11 is a perspective view of the bent rod driving mechanism: the in-line rotor 32 is located in the inlet tube 341 of the hollow housing body 34 (refer to FIG. 16). The in-line rotor 32 has several axial mounting slots 323 which are equally distributed around the cylindrical surface of the in-line rotor 32. The angled rotor 33, with matching axial mounting slots 333, is located in the angled outlet section 342 of the housing body 34 (refer to FIG. 16). A number of bent driving rods 31 (preferably three), of constant diameter and bent at a right or obtuse angle, are positioned with the straight driving segment 311 of each rod contacting the inner wall of the inlet tube 341 of the hollow housing body 34 and the straight driven segment 312 of each rod contacting the inner surface of the outlet wall 351 of the housing head cap 35 (refer to FIG. 17). Each driving rod 31 fits correspondingly inside each mounting slot 323 or 333 of the in-line and angled rotors. The driving rods 31 rotate along the inner surfaces of the dental angle housing 3435 (refer to FIG. 15).

FIG. 12 shows cross-sectional views with side views of the driving mechanism by bent driving rods 31 (shown in FIG. 11 as a perspective view). On the cylindrical surface of the in-line rotor 32 and the angled rotor 33, there are multiple (three preferred) mounting slots 323 and 333 that are equally distributed and parallel to the rotor axis. The cross-section of the mounting slots 323 and 333 is an arc between 180° and 240°. The diameter of the mounting slots 323 or 333 is slightly larger than the diameter of the driving rods 31. When the rods 31 are pressed into the mounting slots 323 and 333, they will fit loosely and slide freely inside the slots 323 and 333. The bent driving rods 31 act as a bearing mechanism between the rotors 31 and 32 and the inner surface of the dental angle housing 3435 (refer to FIG. 15), so that friction is reduced during operation.

Each straight driving segment 311 of a driving rod 31 is radially secured by the inner cylindrical surface of the hollow housing body 34. The straight driving segment 311 sits loosely in the mounting slot 323 of the in-line rotor 32 which itself is positioned by an axial inner tube step 340 of the hollow housing body 34. The housing head cap 35 positions the angled rotor 33 and the bent segment of the driving rod 312. The driving rods 31 here are radially secured by the inner cylindrical surface of the housing head cap 35. The straight driven segment 332 of the driving rod 31 sits loosely in the mounting slot 333 of the angled rotor 33. The angled rotor 33 is positioned by a positioning step 350 in the circular cap ring 354 of the housing head cap 35.

The in-line rotor 32 and the angled rotor 33 have conic heads 322 and 332 and rotor flanges 321 and 331, respectively. The flanges 321 and 331 contact the inlet tube step 340 and angled housing step 350, respectively, thereby preventing the rotors from separating. Similarly, the conic heads 322 and 332 touch each other, thereby maintaining the correct orientation and axial positioning of the rotors 32 and 33.

FIG. 13 shows cross-sectional views of the angled rotor 33 and the rubber cup subassembly 40. The bottom side of the angled rotor 33 is snapped on to the top of the rotor-cup adapter 41. The top end of the rubber cup 42 is pre-assembled or molded on to the bottom end of the rotor-cup adapter 41 to form a disposable, integral assembly 40. The axis of rotation of the rotor-cup adapter 41 and the rubber cup 42 is aligned to the axis of the angled rotor 33. The bottom end of the rubber cup 42 has a skirt opening to receive the mixed prophylaxis and bleaching compounds during operation.

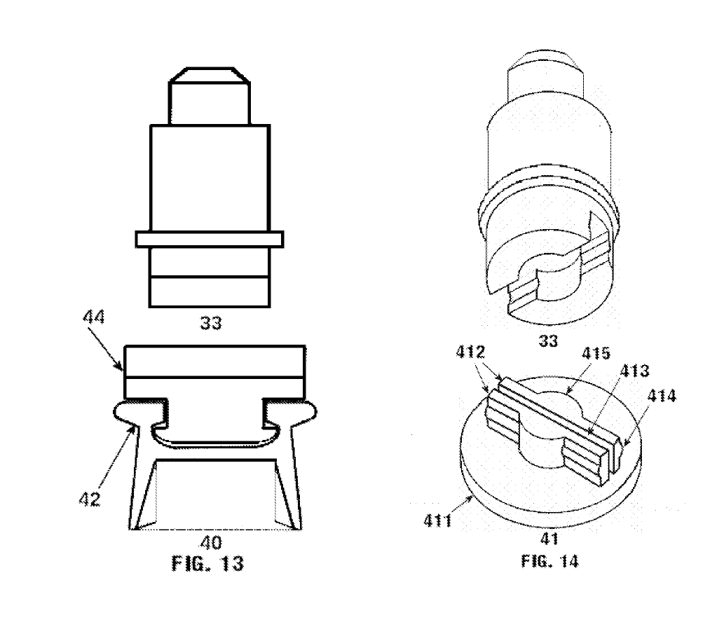

A perspective view of the angled rotor 33 and a different perspective view of the rotor-cup adapter 41 are shown in FIG. 14. The rotor-cup adapter 41 has two protruding ridges 412 that are aligned parallel to the diameter of the adapter base 411. The two ridges 412 are split by a gap 413. Each ridge 412 has a convex ribbed protrusion 414 on the outside of the vertical wall. The ridges 412 also have a thicker center to form a gapped cylindrical positioning drum 415. A negatively matched slot, which forms the counterpart to the entire protruding ridge structure 412, is located in the bottom side of angle rotor 33. The cup assembly 40 (consisting of the rotor-cup adapter 41 and the rubber cup 42; refer to FIG. 13) can therefore be easily pressed into and removed from the bottom end of the angled rotor 33.

FIG. 15 is a perspective view of the dental angle housing 3435. The inner diameter of the input end of the hollow housing body 34 is just slightly larger than the outer diameter of the driving shaft 29 of the driving handpiece 20 for easy insertion during assembling the dental angle 30 to the handpiece 20. The housing head cap 35 is locked on to the hollow housing body 34 by a snap-on lock button connection at the location 34-35 so that after assembly the upper curved buckle hole 353 of the housing head cap 35 matches the curved buckle button 343 of the hollow housing body 34 (refer to FIG. 16).

FIG. 16 is a detailed perspective view of the hollow housing body 34 of the dental angle housing 3435. The housing body 34 comprises an inlet tube 341 and an angled outlet section 342. The housing body bending angle between the axis of the inlet tube 341 and the angled outlet 342 matches the bending angle of the driving rods 31. The angled outlet 342 has a pair of snap-on buckle buttons 343, a bottom step 344, and a vertical open area bounded by the matching wall edges 345. The buckle buttons 343 match the corresponding buckle holes 353 in the housing head cap 35 (refer to the snap-on lock button connection location 34-35 in FIG. 15).

FIG. 17 is a detailed perspective view of the housing head cap 35 of the dental angle housing 3435. The housing head cap 35 has a circular cap ring 354 at its bottom end. After assembly the outlet wall 351 forms a matched part of housing body to match the bending angle of the driving rods 31. The housing head cap 35 has a pair of snap-on buckle holes 353 and a vertical open area bounded by the matching wall edges 355. The buckle holes 353 match the corresponding parts on the housing body 34 when assembled (refer to the snap-on lock button connection location 34-35 in FIG. 15).

The assembly steps for the dental housing subassembly 30 are as follows. First, press the driving rods 31 into the corresponding mounting slots 323 and 333 on the rotors 32 and 33. Second, insert the in-line rotor side of this rod and rotor pre-assembly into the hollow housing body 34. Third, insert the angled rotor side of the pre-assembly into the housing head cap 35, with the bottom end of the angled rotor 33 inside the circular cap ring 354 of the housing head cap 35. Fourth, snap the housing head cap 35 on the housing body 34 to form the complete dental angle 30.

The snap-on locking mechanism of the housing body 34 and the angled cap 35 is achieved by two protruded snap-on buckle buttons 343 near the output end of the inlet tube 341 which match the corresponding snap-on buckle holes 353 near the top of the housing head cap 35. The curved edge wall of the buckle button 343 is a circular arc slightly larger than 180 degrees. The corresponding curved edge wall of the buckle hole 353 has the same arc as the buckle button 343. When the button 343 is snapped into the buckle hole 353, the housing head cap 35 is firmly locked in the housing body 34. The snap-on structure assures that the housing head cap 35 cannot be pulled off by any downward force that may be applied during operation or the cup assembly 40 removal. The circular cap ring 354 is firmly held to the angled outlet 342 at the output end of the hollow housing body 34. Hence, when the bent driving rods 31 drive the angled rotor 33 to rotate along the angled rotor axis, the rods 31 will be held inside the cap ring 354 with minimum wobble, friction, and noise.

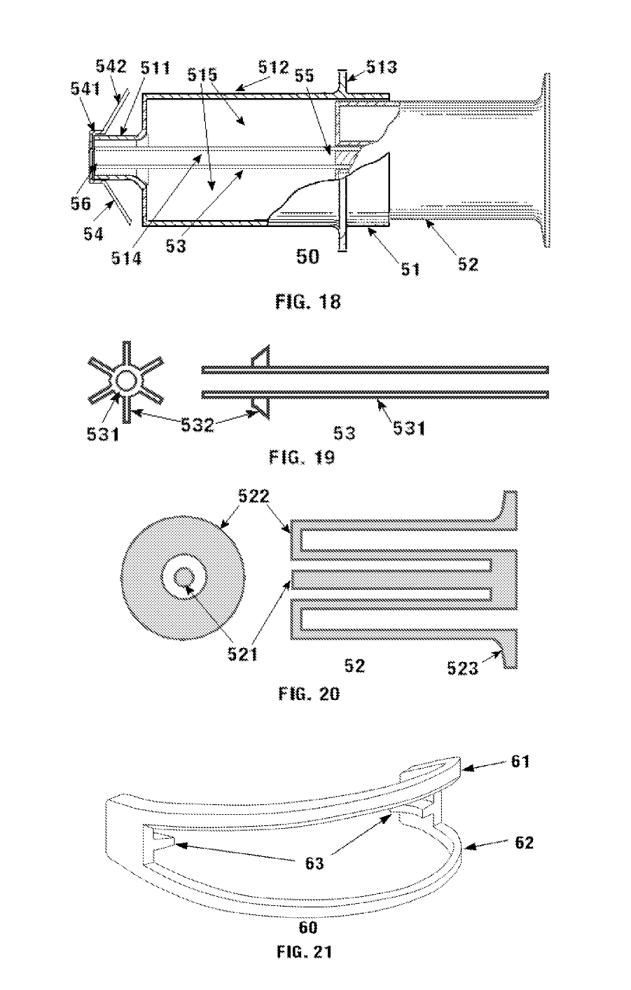

FIG. 18 is a perspective view of the two-component paste dispenser 50. The two components are installed within the central tube 53 and the dispenser housing 51, respectively. A double dispenser piston 52 advances in the central tube 53 and the dispenser housing 51 simultaneously when pressed. The dispenser housing 51 has an open mixing output end 511 at the head of the housing cylinder 512 and an open input end 55 at the tail of the housing cylinder 512. A disposable cover cap 54 seals the mixing output end 56 during storage and serves as a mixing container during use. A dispenser housing flange 513 is located near the open input end 55 of the housing cylinder 512, serving as a finger-grip when the piston 52 is pressed forward. The disposable cover cap 54 consists of a cap cover head 541 which tightly seals the open mixing output end 511 and a cap cover skirt 542 which expends the opening of the cap cover head 541 to facilitate manipulation of the cap cover head 541 during usage.

The central tube 53 divides the space of the dispenser housing 51 into a central reservoir 514, which is defined as the volume inside the central tube 53, and a surrounding shell reservoir 515, which is defined as the volume outside the central tube 53 and inside the dispenser housing 51. In one reservoir 514 or 515, a bleaching peroxide agent with a low pH value is installed. In the other reservoir 515 or 514, an abrasive cleaning prophylaxis agent with a high pH value is installed. Hence the bleaching agent can be stored longer before usage and acts more effectively during usage when proportionally mixed with the prophy agent to increase the pH value and then immediately applied on tooth surfaces.

FIG. 19 shows two cross-sectional views of the dispenser central tube 53. The dispenser central tube 53 consists of a straight tube 531 and tube wings 532 near the output end of the straight tube 531. The location of the tube wings 532 should be matched to the conic mouth near the output end 56 (refer to FIG. 18) so that the content from the reservoir 515 (refer to FIG. 18) is pressed out easily through the slots between the tube wings 532.

Two cross-sectional views of the dispenser piston 52 are shown in FIG. 20. The double piston 52 consists of a central piston 521 of circular cross-section, a surrounding piston 522 of annular cross-section and a piston base at the tail of the piston 52. The circular cross-section of the central piston 521 tightly matches the inner diameter of the central tube 53, and the surrounding annular piston 522 tightly matches the surrounding ring-shaped section of the dispenser housing when the double piston is inserted into the open input end of the dispenser housing 51 (refer to FIG. 18). The dispenser piston base 523, the diameter of which is larger than the diameter of the outer diameter of the surrounding annular piston 522, is used to press both of the central piston 521 and the annular piston 522 simultaneously into the dispenser housing 51 so as to squeeze a proper amount of both the prophy paste and bleaching paste out of the dispenser housing 51 through the open mixing output end 511. The dispenser piston base 523 also is used for a vertical stand when the paste dispenser 50 is not in usage for convenience.

FIG. 21 is a perspective view of the disposable mouthpiece 60. The mouthpiece is oblong in shape. The upper and lower curved sections 61 and 62 engage the tooth and gum surfaces to expose the upper and lower frontal teeth when in use. They abut against the gum surfaces to prevent contact with the bleaching material. At the left and right ends of the upper and lower curved sections 61 and 62, there are wedge-shaped positioning tabs 63. When the user bites down on the tabs, the mouthpiece 60 remains stationary in the mouth with the upper and the lower lips held in a natural open state.

The objectives of the invention are achieved as shown above. Although specific examples of the present invention and its application are set forth herein, they are not intended to be exhaustive or limiting of the invention. These illustrations and explanations are intended to acquaint others skilled in the art with the invention, its principles, and its practical applications, so that others skilled in the art may adapt and apply the invention in its numerous forms, as may best suit the requirements of a particular use.

I claim:

1. A dental prophylaxis and bleaching system comprising: (a) a battery-powered driving handpiece; (b) a dental angle to transfer the rotary oscillation of said driving handpiece through a right or obtuse angle; (c) a disposable rubber cup assembly attached to the distal output end of said dental angle; (d) a disposable dual-component dispenser where prophylaxis and bleaching agents are maintained in separate compartments and mixed at the output, in the correct proportions, when said agents in said dispenser are pressed forwards; and (e) a disposable mouthpiece for protecting the gum surfaces from contact with the bleaching material.

2. The system of claim 1, wherein said driving handpiece comprises:
(a) a battery-powered DC electric motor with an off-axis post on the end face of the motor output shaft; (b) a rotary oscillation driving cam to translate the continuous rotation of said electric motor into a back-and-forth swinging action; (c) a driving shaft to transfer the back-and-forth swinging movement of said rotary oscillation driving cam, via a rack-and-pinion connection, to a rotary oscillation at the output; (d) an light bulb; (e) a power-on-off switch to connect the light bulb and the motor to the batteries; and (f) a handpiece housing to enclose all internal parts of said driving handpiece.

3. The driving handpiece of claim 2, wherein said rotary oscillation driving cam, being mounted onto a cam pivot post that is part of inside of said handpiece housing, has a cam curvilinear slot in its mid-section, within which said off-axis post of said DC motor is confined, thereby converting the continuous rotation of said off-axis post into a back-and-forth swinging action, and a rack gear on the side facing away from said DC motor.

4. The rotary oscillation driving cam of claim 3, wherein said cam curvilinear slot, in order to more closely balance the speed and duration of the rightward and leftward motions of the back-and-forth swinging action, is curved such that when said off-axis post on said DC electric motor moves clockwise, starting from the position farthest from the cam pivot post, herein referred to as the top, down a quarter of circle, said driving cam swings to the right, about said cam pivot post, pausing briefly where the curvature of said slot matches the curvature of the right side of the circle traced by the motion of said off-axis post, and such that when said off-axis post continues to move down another quarter of circle to the bottom point and then continues to move up another quarter of circle, said driving cam swings to the left, about said cam pivot post, again pausing briefly where the curvature of said slot matches the curvature of the left side of the circle traced by the motion of said off-axis post, and such that when said off-axis post continues to move clockwise up another quarter of circle to the top, said driving cam finishes one cycle.

5. The rotary oscillation driving cam of claim 3, wherein said off-axis post on said DC electric motor moves counterclockwise, starting from the position farthest from said cam pivot post down a quarter of circle, said driving cam swings to the left, about said cam pivot post, pausing briefly where the curvature of said slot matches the curvature of the left side of the circle traced by the motion of said off-axis post, and such that when said off-axis post continues to move down another quarter of circle to the bottom point and then continues to move up another quarter of circle, said driving cam swings to the right, about said cam pivot post, again pausing briefly where the curvature of said slot matches the curvature of the right side of the circle traced by the motion of said off-axis post, and such that when said off-axis post continues to move counterclockwise up another quarter of circle to the top, said driving cam finishes one cycle.

6. The rotary oscillation driving cam of claim 3, wherein said cam curvilinear slot is curved such that when the curvature of said slot matches the curvature of the right or left side of the circle traced by the motion of said off-axis post, said rotary oscillation driving cam experiences a momentary pause in its back-and-forth swinging action, thereby reducing the impulse from said motor shaft off-axis post to said rotary oscillation driving cam as the back-and-forth swinging action changes direction.

7. The driving handpiece of claim 2, wherein said driving shaft has a pinion gear at its input end, matching said rack gear on said driving cam, as well as a matched slot-to-rod connection at its output end, so as to transfer the back-and-forth swinging action of said driving cam to a rotary oscillation at the input end of said dental angle.

8. The driving handpiece of claim 2, wherein said light bulb is mounted at a proper location on said handpiece housing so that its light, which is focused at the proper distance, is always emitted towards the working area of said dental prophy angle thereby serving as an illumination source as well as heating up said bleaching material in order to speed up the activation of bleaching.

9. The system of claim 1, wherein said dental angle comprises: (a) an axially-rotating in-line rotor, (b) an axially-rotating angled rotor, (c) multiple bent driving rods, bent at a right or obtuse angle, consisting of a straight driving segment on one side of the bend and a straight driven segment on the other side of the bend, by which the rotary oscillation is transmitted from said in-line rotor to said angled rotor; and (d) a dental angle housing which, after assembly, locks said in-line rotor and said angled rotor into one integral piece by means of steps in said housing and corresponding flanges on said rotors.

10. The dental angle of claim 9, wherein said dental angle housing consists of two sections to facilitate assembly; firstly, a hollow housing body, which is placed over the output end of said driving shaft of said driving handpiece into where said in-line rotor and said multiple driving rods are positioned; and, secondly, a housing head cap, which is placed over said angled rotor and which mates with the bent end of said housing body to form the complete housing entity.

11. The dental angle housing of claim 10, wherein said housing head cap has a circular ring at its lower end which positions said angled rotor and said bent driving rods properly during operation, as well as a pair of button-hole snap-on buckles at its upper end which lock said housing head cap to said housing body when assembled.

12. The driving shaft of claim 7, wherein said matched slot-to-rod connection consists of several slots in the conic nose end of said driving shaft, into which are inserted, at the time of assembly, said bent driving rods of said dental angle, and wherein the diameter of said slots is gradually and smoothly enlarged toward said conic nose end thereby avoiding any possible slot and rod mismatch.

13. The dental angle of claim 9, wherein both said in-line and angled rotors have similar axial mounting slots equally distributed around their cylindrical surface and said mounting slots have a circular cross-section with a diameter slightly larger than that of said bent driving rods allowing said driving rods to slide freely within said mounting slots, and wherein the depth of said mounting slots below the cylindrical surface of said rotors is slightly less than the diameter of said bent driving rods so that said bent driving rods contact the inner cylindrical surface of said housing body and said housing head cap thereby acting as a bearing mechanism to reduce the friction between said bent driving rods and the inner cylindrical surface of said housing body and head cap during operation.

14. The bent driving rods of claim 13, the diameter of which is slightly larger than the opening width of said mounting slots on said in-line rotor and said angled rotor so that said bent driving rods can be easily pressed into the side of said mounting slots and be retained inside, thereby avoiding the time-consuming process of inserting said bent driving rods through the end of said mounting slots.

15. The dental angle of claim 9, wherein said in-line rotor and said angled rotor both have a conic head and an axial positioning step such that after assembly said conic heads contact each other and such that both rotors are held in the proper axial position.

16. The dental angle of claim 9, wherein said bent driving rods are bent at a right or an obtuse angle with respect to said in-line rotor axis, exactly matching the angle of said dental angle housing, such that after assembly the axis of said straight driving segment of each bent driving rod is parallel to the axis of the corresponding mounting slot of said in-line rotor and said straight driven segment of each bent driving rod is parallel to the axis of the corresponding mounting slot of said angled rotor, allowing said bent driving rods to simultaneously rotate and axially slide within said mounting slots of said rotors during operation.

17. The system of claim 1, wherein said disposable rubber cup assembly comprises: (a) a ribbed, disk-shaped rotor-cup adapter that has two protruding ridges on the top, said ridge being aligned the diameter with a horizontal split along its center line, and said ridge having a cylindrical vertical positioning drum at the center, and the horizontal walls of said ridge having a convex ribbed protrusion running along their full length, all of which elements allow said rotor-cup adapter to be firmly snapped into a slot in the end of said angled rotor such that the axis of rotation of said rubber cup assembly is aligned with that of said angled rotor, and (b) a rubber cup which receives said prophylaxis and bleaching agents from said dual-component dispenser.

18. The system of claim 1, wherein said dual-component dispenser comprises: (a) a dispenser housing with an open mixing output end at the head and an open input end at the tail; (b) a central tube co-axially parallel to said dispenser housing; (c) a double piston consisting of a central piston of circular cross-section, which matches the cross-section of said central tube, and a surrounding piston of annular cross-section, which matches the cross-section of the remainder of said dispenser housing, and a platform at the tail end upon which to press said double piston into said dispenser housing; and (d) a cover/mixing cap which seals said mixing output end while in storage and can be used disposably as a mixing plate, in conjunction with said rubber cup after said component pastes are pressed out into said cap.

19. The dual-component dispenser of claim 18, wherein said central tube divides the volume of said dispenser housing into a central reservoir and a surrounding shell reservoir, where an abrasive cleaning prophylaxis agent with a high pH value and a bleaching peroxide agent with a low pH value are stored separately in each reservoir, such that the separate storage extends the life of said bleaching agent and increasing its effectiveness by delaying the moment of mixture with said prophylaxis agent until immediately before applying the mixture to the tooth surfaces.

20. The system of claim 1, wherein said disposable mouthpiece has upper and lower sections curved so as to cover the user's gum surfaces to prevent contact with the bleaching material while the upper and lower frontal teeth are cleaned, as well as a wedge-shaped bite tab extending inward from both of the two points of juncture of said upper and lower curved sections, so that when the user bites down on said bite tabs, said disposable mouthpiece remains stationary within the mouth, and the upper and lower lips are held apart at the proper jaw angle.
